(19) United States
(12) Patent Application Publication
(10) Pub. No.: US 2024/0424218 A1
(43) Pub. Date: Dec. 26, 2024

(54) VIAL-TO-SYRINGE CONVERTER AND METHODS OF MAKING AND USING SAME

(71) Applicant: Merck Sharp & Dohme LLC, Rahway, NJ (US)

(72) Inventors: Daniel A. Bonanno, Rahway, NJ (US); Eduardo Cristofolli, Rahway, NJ (US); Cassie Megna, Rahway, NJ (US); Wail A. Rasheed, Rahway, NJ (US); Ophelia L. Wells, Easton, PA (US)

(73) Assignee: Merck Sharp & Dohme LLC, Rahway, NJ (US)

(21) Appl. No.: 18/701,306

(22) PCT Filed: Nov. 1, 2022
(86) PCT No.: PCT/US2022/048565

§ 371 Date: Apr. 15, 2024

Related U.S. Application Data
(60) Provisional application No. 63/275,106, filed on Nov. 3, 2021.

(51) Int. Cl. A61M 5/24; A61J 1/14; A61J 1/20

(52) U.S. Cl. CPC A61M 5/2429; A61J 1/1468; A61J 1/201; A61J 1/2096

(57) ABSTRACT

In some examples, a vial-to-syringe converter includes a plunger rod, an adapter including a body having a piercing spike, a receiver and a lumen extending between the spike and the receiver, the body further including at least one laterally-extending flange, and a needle in communication with the receiver and configured to deliver a medicament to a patient's body.

### FIELD OF THE DISCLOSURE

The present disclosure relates generally to syringes and injectors. More specifically, the present disclosure relates to vial-to-syringe converters for container closure devices.

### BACKGROUND OF THE DISCLOSURE

Prefilled syringes and cartridges are highly accurate parenteral devices used for the delivery of many vaccines, biologics, therapies, and the like. Parenteral products contain many advantages such as minimizing drug waste and increasing product life span. Additionally, parenteral devices are easily recognized by healthcare workers as a convenient method of drug delivery. One disadvantage to these parental devices is that they are typically introduced later in the drug development process and require long, complex procedures to satisfy various agency requirements through clinical trials. This, for examples, includes having a better understanding of all components, materials and interactions needed in the container closure system that creates the parenteral delivery. This process can delay critical drugs to clinic and to market.

Thus, there exists a need for devices that improve upon and advance the methods of safely bridging the gap between early drug development phases and later drug development phases.

### SUMMARY OF THE DISCLOSURE

In one embodiment, a system of creating a vial with a luer-lock functional area for use as a syringe (vial-syringe adaptor) and used as a convenient method of drug delivery. In some embodiments, vial-to-syringe converter includes a plunger rod, an adapter including a body having a piercing spike, a receiver and a lumen extending between the spike and the receiver, the adapter further including at least one laterally-extending flange, and a needle in communication with the receiver and configured to deliver the medicament to a patient's body.

In some embodiments, a system of delivering a medicament includes a vial-to-syringe converter including a plunger rod, an adapter including a body having a piercing spike, a receiver and a lumen extending between the spike and the receiver, the adapter further including at least one laterally-extending flange, and a needle in communication with the receiver and configured to deliver the medicament to a patient's body, and a vial comprising a borosilicate glass body defining a chamber, a stopper disposed inside the glass body and translatable relative thereto, and a septum disposed opposite the stopper.

In some embodiments, a system of delivering a medicament includes a vial-to-syringe converter including a plunger rod, an adapter including a body having a piercing spike, a receiver and a lumen extending between the spike and the receiver, the adapter further including at least one laterally-extending flange, and a needle in communication with the receiver and configured to deliver the medicament to a patient's body, and a vial comprising a borosilicate glass body, a first stopper disposed inside the glass body and translatable relative thereto, a second stopper disposed inside the glass body and translatable relative thereto, the second stopper being spaced from the first stopper and defining a first chamber on one side and a second chamber on an opposite side, a septum disposed opposite the stopper, and a bypass disposed adjacent the second stopper.

In some embodiments, a system of delivering a medicament includes a vial-to-syringe converter for use in single dose vials or multidose vials. In some embodiments, a system of delivering a medicament includes a vial-to-syringe converted for use in liquid, liquid-liquid, and liquid-dry products.

### BRIEF DESCRIPTION OF THE DISCLOSURE

Various embodiments of the presently disclosed syringe assemblies and converters are disclosed herein with reference to the drawings, wherein:

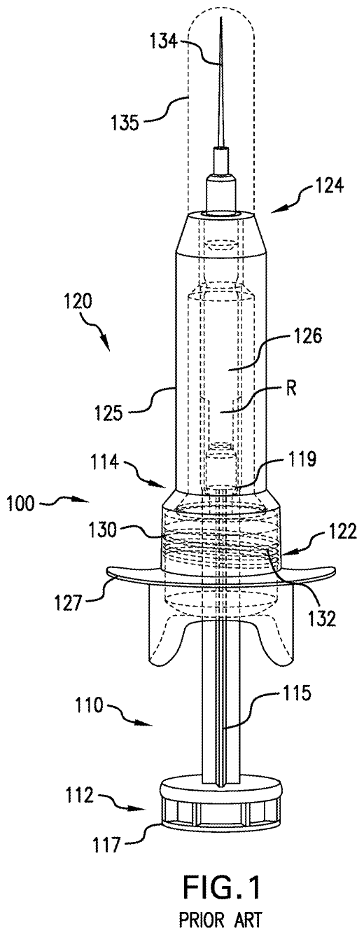

FIG. 1 is a schematic front view of a pre-filled syringe:

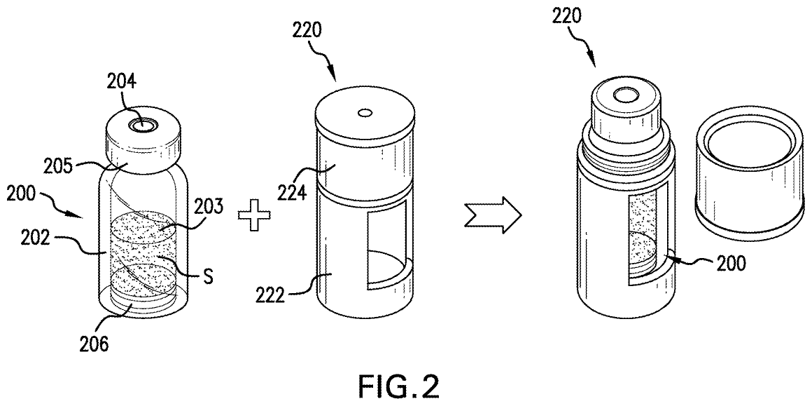

FIG. 2 is a schematic perspective view of a vial and a vial shell according to one embodiment of the disclosure:

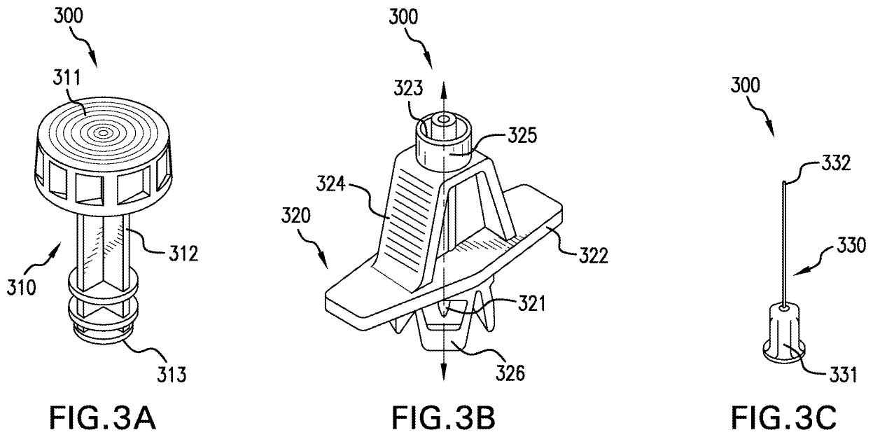

FIGS. 3A-C are schematic perspective views of a plunger rod, an adapter, and a needle according one embodiment of the disclosure:

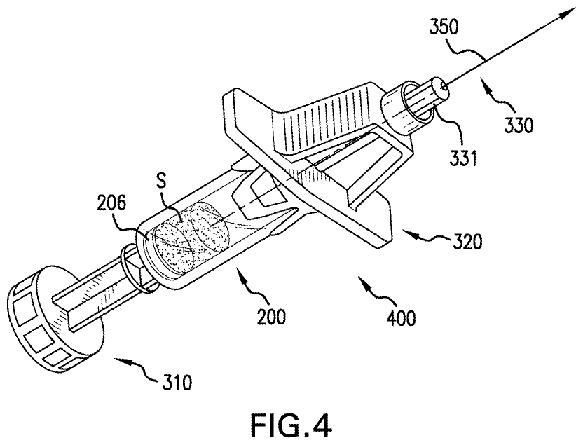

FIG. 4 is a schematic perspective view of a functional syringe assembly having the vial of FIG. 2 according one embodiment of the disclosure:

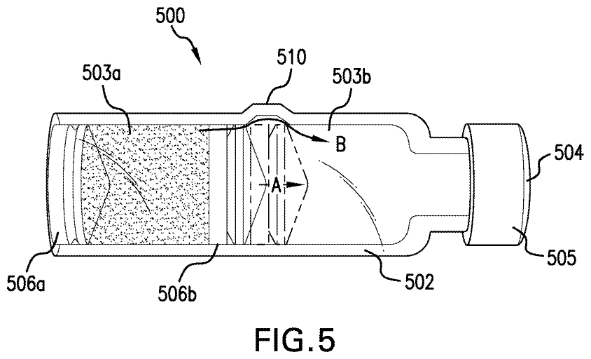

FIG. 5 is a schematic perspective view of another embodiment of a vial according one embodiment of the disclosure: and

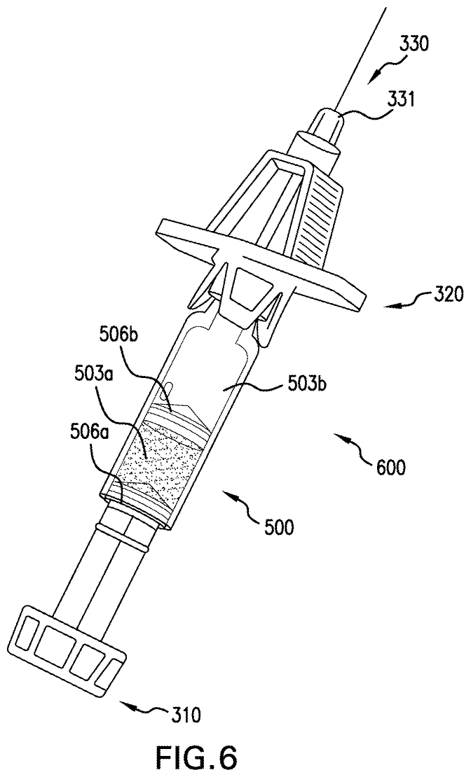

FIG. 6 is a schematic perspective view of a functional syringe assembly having the vial of FIG. 5 according one embodiment of the disclosure.

Various embodiments will now be described with reference to the appended drawings. It is to be appreciated that these drawings depict only some embodiments of the disclosure and are therefore not to be considered limiting of its scope.

### DETAILED DESCRIPTION

Despite the various improvements that have been made to container closure devices, injectors and syringes, such as pre-filled syringes, conventional methods suffer from some shortcomings as discussed above.

Therefore, there is a need for further improvements to the devices and methods used to deliver medication. Among other advantages, the present disclosure may address one or more of these needs.

As used herein, the term “proximal,” when used in connection with a component of a syringe or injector, refers to the end of the component closest to the user's hands when holding the device, whereas the term “distal,” when used in connection with a component of a syringe or injector, refers to the end of the component closest to the needle insertion site during use.

Likewise, the terms “trailing” and “leading” are to be taken as relative to the operator's fingers (e.g., physician) of the syringe or injector. “Trailing” is to be understood as relatively close to the operator's fingers, and “leading” is to be understood as relatively farther away from the operator's fingers.

Reference is now made to FIG. 1, which shows an exemplary prefilled-syringe 100 contained within a needle safety device having two states, a first state with the needle extended before injection (FIG. 1), and a second state with the needle retracted within a barrel after the full injection has been completed. It will be understood that though a needle within a safety device is shown, the disclosure is not thus limited. For example, sensors of the present disclosure may be integrated into a specially designed syringe barrel or through the use of a separate assembly that could be attached to the syringe prior to use. Additionally, though a pre-filled syringe with a staked needle is shown, it will be understood that the principles disclosed herein are equally applicable to other types of injectors (e.g., syringes with removable needles, auto-injectors, or on-body (wearable) injectors, etc.). Pre-filled syringe 100 generally comprises two main portions, a plunger rod 110 and a barrel 120. Plunger rod 110 generally extends between a proximal end 112 and a distal end 114 and comprises an elongated piston 115 extending between a plunger flange 117 and a coupler 119. In one embodiment, piston 115 has a cruciform cross-sectional shape.

A cylindrical barrel 120 extends between proximal end 122 and distal end 124 and comprises a body 125 defining a lumen 126 for accepting a portion of plunger rod 110. Body 125 further comprises a barrel flange 127 adjacent proximal end 122 and defines a reservoir “R” that holds a medicament, drug, saline, or other substance for injecting into a patient's body. An internally threaded stopper 130 is disposed inside lumen 126 of body 125. In one embodiment, stopper 130 is made of an elastomeric material such as natural rubber, synthetic rubber, thermoplastic elastomers, or combinations thereof, and comprises an opening to receive and mate with coupler 119 of plunger rod 110 by advancing the plunger rod inside the barrel lumen 126 and rotating at least one of coupler 119 and stopper 130 relative to the other.

In this example, pre-filled syringe 100 includes a spring 132 operatively coupled to needle 134 to provide an additional safety mechanism. A cap 135 is also disposed over needle 134. Once cap 135 is removed, the user may pierce the patient's skin with the needle, then push on plunger flange 117 to drive the plunger to deliver a medicament through needle 134 into the patient's body. Spring 130 is configured so that, upon actuation and full delivery of the medicament, needle 134 will safely retract within barrel 120 and be locked inside to reduce the risk of needlestick injuries.

In some embodiments, a vial and a converter combination may be used instead of a traditional pre-filled syringe that requires regulatory approval. Turning to FIG. 2, vaccines and biologics may be stored in vials 200 made of borosilicate glass, known for its chemical stability and its ability to withstand long refrigeration and impact during transportation. Additionally, borosilicate glass has a low likelihood of chemical leaching. The vials 200 may include a glass body 202 and a movable elastomeric stopper 206, disposed on one end and translatable within the glass body 202. In one embodiment, the material of the elastomeric stopper 206 comprises any elastomeric material similar to syringe stoppers with same material properties currently in use in the market. In a further embodiment, the elastomeric stopper comprises any elastomeric material that can create a seal and have relative motion with contact container 222.

Body 202 itself defines a main chamber 203 for receiving a substance “S” or medicament (e.g., vaccine, biologic, therapeutic, drug product for use in early drug development phases, etc.), and a latex septum 204 having a circumferential aluminum crimp 205 disposed on an opposite end. Vials 200 may be protected inside shell 220 that includes a body 222 and a protective cap 224. Shell 220 may be useful for ease of labeling when the drug products requires conditioning or storage in conditions outside of a standard range. In one embodiment, the shell comprises any medical grade plastic and/or polymer that is presently on the market. In a further embodiment, the shell comprises polypropylene.

As shown in FIGS. 3A-D, a converter system 300 may be used to transform the vial 200 into an operable syringe assembly. The converter system 300 generally includes a plunger rod 310, an adapter 320 and a needle 330, the three components being operable to be joined with the vial 200 to form a syringe assembly.

Plunger rod 310 may generally include an actuating base 311, an elongated cruciform shaft 312 and a vial contacting member 313. As shown, actuating base 311 may be substantially circular and enlarged relative to the shaft and the contacting member so that a user can press on it with their thumb or other digit. In some examples, plunger 310 is formed of a unitary body made of any medical grade plastic and/or polymer that is presently on the market. In one embodiment, the plunger comprises polypropylene.

Adapter 320 may also be formed of any medical grade plastic and/or polymer that is presently on the market. In one embodiment, adapter comprises polypropylene. Adapter 320 may include a piercing device/apparatus 321 (a feature that creates an access from vial i.e. a fluid path), a body 324 defining a pair of finger flanges 322, and a needle receiver 323 disposed opposite the spike 321. The adapter 320 may define an axial lumen 325 that extends from the piercing spike 321 through the body and the receiver 323. Optionally, a plurality of wings 326 may be circumferentially disposed about the piercing spike 321 and configured to accept and/or stabilize a portion of a vial. Needle receiver 323 may be sized to accept a hub 331 of a needle 330 so that the hub 331 can be pressed or twisted into the receiver 323 to mate the two components together and establish a passage for fluid communication from spike 321 of adapter 320 to the distal tip 332 of needle 330. In at least some examples, needle 330 may be a conventional luer-lock needle, and the receiver 323 includes an internal threading that complements the luer-lock threading of the needle hub.

Turning to FIG. 4, the converter system 300 is shown as it is coupled to a vial 200 to turn it into a functional syringe assembly 400. Specifically, plunger rod 310 may be connected to one end of a vial 200, and specifically the stopper 206 of vial 200 (e.g., through threaded engagement, friction fitting, or other suitable technique). In at least some examples, plunger rod 310 is disposed at least partially within body 202 and pressed against stopper 206. On the opposite end, piercing spike 321 may be used to perforate the septum of the vial so that the contents of the vial can flow through the spike 321. Needle 330, and particularly hub 331, can be connected via compression or threaded engagement to receiver 323 of adapter 320 to create a continuous passage which directs the fluid from the vial through the needle into the patient with the application of pressure to the plunger rod.

To use the system, a clinician, patient, or user may assemble the components as described above, or the components may be pre-assembled by a manufacturer or care provider. The distal end of needle 330 may be pierced into a body part (e.g., the patient's arm). To deliver the drug product into the patient, the user may simply grasp the finger flanges 322 of adapter 320 with, for example, their index and middle fingers, and press against the plunger rod 310 with their thumb so that the plunger rod pushes the stopper 206 of the vial 200 through the body 202, and the contents of the main chamber 203 flow through passageway 350 (i.e., through the spike, the adapter body, the hub of the needle and the distal end of the needle) into the patient's body.

Thus, a single vial 200 may be used in different phases of development while minimizing regulatory and safety risks. In this manner, the converter provides a simple, easy-to-use method to accurately deliver a single dose from a custom vial 200 and to transform the vial into an easy-to-recognize parental syringe assembly. Among the advantages of this configuration are the benefits of providing a way for patients to self-administer at home, reducing environmental waste, reducing development time, decreasing time to market, and improving access to underserved markets. Additionally, the present embodiments reduce the need to overfill a drug product to account for waste during withdrawal, which is beneficial for programs or stages where drug availability is limited.

In another embodiment, a dual-chamber vial 500 may include a glass body 502, a latex septum 504 and a crimp 505 similar to that of FIG. 2. In the embodiment of FIG. 5, vial 500 defines a first chamber 503a, a second chamber 503b, a first stopper 506a, and a second stopper 506b. Each of the two stoppers 506a,506b may be axially translatable inside the body 502, and the first chamber 503a may be defined between the two stoppers. Second chamber 503b may be defined between second stopper 506b and the latex septum. In at least some examples, the two chambers are of equal size and configured to hold an equal volume of substances. In a resting state, the second stopper 506b may form a fluid-tight partition between the two chambers. A flared bypass 510 is formed on a sidewall of the body between the two chambers, the flared bypass 510 including a bulge in the body (i.e., the body has greater inner and outer diameters at the bypass). The dual-chamber vial 500 utilizes the bypass 510 as a path for the contents of the first chamber 503a to ingress into the second chamber 503b with the application of pressure to a plunger rod following the engagement into the vial's plunger stopper and displacement of the second stopper 506b as shown by arrow “A”. In at least some examples, the bypass is disposed adjacent a midpoint of the glass body (i.e., at or near a point halfway along the length of the glass body). Displacing the second stopper 506b to a second position adjacent bypass 510 may remove the fluid-tight partition between the two chambers and open a passageway between the two chambers so that contents of the first chamber may flow into the second chambers as shown by arrow “B”, and together they may be delivered out of the needle.

In some examples, the two chambers contain a two-drug product combination so that two different substances may be stored separately and introduced together. For example, a first liquid may be disposed in the first chamber, and a second liquid may be disposed in the second chamber. Alternatively, a liquid may be disposed in the first chamber, and a lyophilized product may be disposed in the second chamber. In at least some examples, the first chamber may contain a first pharmaceutical composition or drug substance, and the second chamber may contain a second pharmaceutical composition or drug substance. In at least some examples, the first chamber may contain any of any injectable product (lyophilized product, saline, water, drug product, medicine), and the second chamber may contain any of an injectable product that can be used in combination of chamber 1 for investigative, therapeutic, or medicine use. In one embodiment, the first chamber may contain any of one injectable product, and the second chamber may contain a different injectable product. In a further embodiment, the first and second chamber comprise injectable medical products for use in a combination.

In another embodiment, the first and second chamber comprise the same injectable medical product for use as a single administration to achieve a desired dose.

A converter system 300 may be coupled to a vial 500 to turn it into a functional syringe assembly 600 in a manner similar to that of FIG. 4. Specifically, plunger rod 310 may be connected to one end of a vial 500, and specifically the first stopper 506a using any of the engagement techniques previously described. In at least some examples, plunger rod 310 is disposed at least partially within body 502 and pressed against stopper 506a. Piercing spike 321 may be used to perforate the septum 504 of the vial on the opposite end so that the contents of the vial can flow through the spike 321. Needle 330, and particularly hub 331, can be connected via compression or threaded engagement to receiver 323 of adapter 320 to create a continuous passage which directs the fluid from the vial through the needle into the patient with the application of pressure to the plunger rod. As plunger rod 310 is advanced forward, the contents of the first chamber will be urged forward, and displace the second stopper 506b, moving the second stopper 506b adjacent bypass 510, and opening a passageway between the two chambers so that contents of the first chamber may flow into the second chambers, and together they may be delivered out of the needle.

It is to be understood that the embodiments described herein are merely illustrative of the principles and applications of the present disclosure. For example, the number, positioning and arrangement of bypass may be varied. Additionally, more than two compartments arranged in parallel may be formed. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A vial-to-syringe converter comprising:
a plunger rod:
an adapter including a body having a piercing spike disposed at a first end, a receiver disposed at a second end opposite the first end, and a lumen extending between the spike and the receiver, the body further including at least one laterally-extending flange; and
a needle in communication with the receiver and configured to deliver a medicament to a patient's body.

2. The vial-to-syringe converter of claim 1, wherein the plunger rod includes an actuating base, an elongated shaft, and a vial contacting member.

3. The vial-to-syringe converter of claim 1, wherein the at least one laterally-extending flange includes two flanges.

4. The vial-to-syringe converter of claim 1, wherein the adapter further comprises a plurality of circumferentially extending wings disposed about the piercing spike.

5. The vial-to-syringe converter of claim 1, wherein the needle comprises a hub coupleable with the receiver of the adapter.

6. The vial-to-syringe converter of claim 5, wherein the hub and the receiver comprise a luer fitting.

7. The vial-to-syringe converter of claim 1, further comprising a passageway providing fluid communication between the piercing spike and the distal end of the needle.

8. A system of delivering a medicament comprising:
a vial-to-syringe converter including a plunger rod, an adapter including a body having a piercing spike disposed at a first end, a receiver disposed at a second end opposite the first end, and a lumen extending between the spike and the receiver, the body further including at least one laterally-extending flange, and a needle in communication with the receiver and configured to deliver a medicament to a patient's body; and
a vial comprising a borosilicate glass body defining a chamber, a stopper disposed within the glass body and translatable relative thereto, and a septum disposed opposite the stopper.

9. The system of claim 8, wherein the plunger rod of the vial-to-syringe converter is coupleable to the stopper of the vial.

10. The system of claim 8, wherein the piercing spike of the vial-to-syringe converter is engageable with a septum of the vial.

11. The system of claim 8, wherein the vial is disposed between the plunger rod and the adapter of the vial-to-syringe converter.

12. The system of claim 8, wherein the plunger rod includes an actuating base, an elongated shaft, and a vial contacting member.

13. The system of claim 8, wherein the at least one laterally-extending flange includes two flanges.

14. The system of claim 8, wherein the adapter further comprises a plurality of circumferentially extending wings disposed about the vial when the vial is coupled to the adapter.

15. The system of claim 8, wherein the chamber of the vial is in fluid communication with the needle when the vial, the adapter and the needle are coupled together.

16. A system of delivering a medicament comprising:
a vial-to-syringe converter including a plunger rod, an adapter including a body having a piercing spike, a receiver and a lumen extending between the spike and the receiver, the body further including at least one laterally-extending flange, and a needle in communication with the receiver and configured to deliver a medicament to a patient's body; and
a vial comprising a borosilicate glass body, a first stopper disposed within the glass body and translatable relative thereto, a second stopper disposed inside the glass body and translatable relative thereto, the second stopper being spaced from the first stopper and defining a first chamber on one side and a second chamber on an opposite side, a septum disposed on one side of the glass body, and a bypass disposed adjacent the second stopper.

17. The system of claim 16, wherein the bypass includes a bulge in the glass body.

18. The system of claim 16, wherein the bypass is disposed approximately halfway along a length of the glass body.

19. The system of claim 16, wherein the first chamber and the second chamber are equal in volume.

20. The system of claim 16, wherein the bypass allows fluid communication between the first chamber and the second chamber when the second stopper is displaced.
